(19) United States
(12) Patent Application Publication
(10) Pub. No.: US 2005/0215895 A1
(43) Pub. Date: Sep. 29, 2005

(54) Devices and methods for obtaining three-dimensional images of an internal body site

(76) Inventors: Richard L. Popp, Los Altos Hills, CA (US); Ali H. Hassan, Stanford, CA (US); Christian S. Eversull, Palo Alto, CA (US); Jeremy Johnson, San Francisco, CA (US); Paul G. Yock, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/985,656

(22) Filed: Nov. 10, 2004

Related U.S. Application Data
(60) Provisional application No. 60/519,557, filed on Nov. 12, 2003.

(51) Int. Cl. A61B 8/00; A61B 8/12; A61B 8/14

(52) U.S. Cl. 600/437

(57) ABSTRACT

Devices and methods for obtaining a three-dimensional image of an internal body site are provided. The subject devices are elongated structures (e.g., catheters) having a plurality of ultrasonic transducers located at their distal end. The configuration of the plurality of ultrasonic transducers may be reversibly changed from a first to a second configuration, where the radial aperture of the plurality of ultrasonic transducers is greater in the second configuration than in the first configuration. A feature of certain embodiments of the subject invention is that the plurality of ultrasonic tranducers are configured in the second configuration as a substantially continuous set of transducers. In using the subject imaging devices, the distal end of the devices is positioned at the internal body site of interest while the plurality of ultrasonic transducers is in the first configuration. The configuration of the ultrasonic transducers in then changed to the second configuration for imaging the internal body site. The subject devices and methods for their use find application in imaging a variety of different internal body sites.

### CROSS-REFERENCE TO RELATED APPLICATIONS

Pursuant to 35 U.S.C. § 119 (e), this application claims priority to the filing date of the U.S. Provisional Patent Application Ser. No. 60/519,557 filed Nov. 12, 2003, the disclosure of which is herein incorporated by reference.

### INTRODUCTION

Background of the Invention

Presently, minimally invasive devices are employed in the treatment of relatively large visceral cavities such as the heart, blood vessels, organs of the abdominal cavity, the urogenital tract, the brain, etc. Catheters have been developed for ablation of tissue, for treatment of arrhythmia, coronary heart disease, placement of devices to treat congenital heart diseases, valvular heart diseases, and congestive heart diseases.

All the above procedures require reliable visualization of the treatment device with reference to the actual position within the body and/or and the target region within the organ. Current visualization techniques include x-ray fluoroscopic imaging, which provides planar two-dimensional (2-D) imaging showing the catheter within the body. However, the nature of x-ray imaging does not allow soft-tissue differentiation. Also, x-ray computed tomography and magnetic resonance imaging techniques do not support real-time three-dimensional (3-D) viewing of the heart and other structures to enable precise guidance of the procedure within the viewed structures.

With respect to optical imaging methods, these imaging methods also have limitations in that optical imaging methods show only the interior surface of a bodily cavity, in which the fiberoptic device is placed. Structures beneath this surface are not perceived.

Ultrasound has proven to be a powerful tool for imaging parts of the body because of its ability to discriminate various soft-tissues based on their ultrasound characteristics (intensity). Ultrasound is a tomographic imaging tool, with routine applications in medicine that provides 2-D images. Ultrasound imaging can be transcutaneous with good far-field resolution depicting bodily structures in 2-D images.

Recently, transcutaneous 3-D applications were introduced to provide volumetric images of the heart, and its chambers. Disadvantages of transcutaneous 3-D imaging include inferior performance under conditions of unfavorable anatomy of the chest wall, air filled gut and lungs, reduced structure resolution at increased distances from the ultrasound source, bulky transducer sizes, and the requirement that the physician conducting minimally-invasive treatment be guided by an image interpretation person.

Alternatively, catheter-based ultrasound has been clinically introduced. This technology has a good near-field resolution. Its proximity to heart structures and bypassing chest wall barriers allows for good resolution. Current ultrasound catheter designs include: (1) single-element transducer crystals that are pointed radially outward and rotated about the axis of the catheter; (2) radial phased array transducers; and (3) linear array transducers. A disadvantage of may ultrasound catheter configurations known to the inventors is that they provide only 2-D information of the region examined by the catheter.

Attempts have been made to construct 3-D images using a catheter with a linear ultrasonic array by collecting multiple 2-D image data fames. In such applications, multiple 2-D images are collected using the array mounted on the catheter, and the collected images are coupled with relative positional information among the image frames so that these image frames may be subsequently assembled into a 3-D volume to form the desired 3-D reconstruction. The relative positional information is acquired by externally rotating the catheter while trying to maintain angular control. Such manual techniques are generally slow and cumbersome.

Another approach to generate volumetric ultrasound images is described by U.S. Pat. No. 5,876,345 (Eaton et al). This document suggests a catheter with at least two linear ultrasound transducer arrays (linear and radial). Sequential imaging is performed using one single ultrasound array at a time, automatically reconstructing volumetric data based on 2-D information obtained from the above two planes (linear and radial). However, a major limitation of this design is a requirement to keep catheter introduction diameter at a reasonable low dimension.

As such, there is still a need for improved 3-D imaging methods and devices for practicing the same. Of particular interest would be the development of an ultrasound imaging device, e.g., catheter, which—during operation—provides a large radial aperture to produce an adequately wide imaging plane perpendicular to the catheter axis, but also has a low catheter profile during introduction into the body site to be imaged. The present invention satisfies this need.

Relevant Literature

US patents of interest include: U.S. Pat. Nos. 6,494,843; 6,482,162; 6,306,096; 6,171,247; 6,162,175; 6,129,672; 6,099,475; 6,039,693; 5,876,345; 5,848,969; 5,713,363; 5,325,860. Also of interest are published United States patent application Ser. Nos. 2002/0026118 and 2202/0049383.

### SUMMARY OF THE INVENTION

Devices and methods for obtaining a three-dimensional (3-D) image of an internal body site are provided. The subject devices are elongated structures (e.g., catheters) having a plurality of ultrasonic transducers located at their distal end. The configuration of the plurality of ultrasonic transducers may be reversibly changed from a first to a second configuration, where the radial aperture of the plurality of ultrasonic transducers is greater in the second configuration than in the first configuration. In certain embodiments, the plurality of ultrasonic tranducers are configured in the second configuration as a substantially continuous set of transducers. In using the subject imaging device, the distal end of the device is positioned at the internal body site of interest while the plurality of ultrasonic transducers is in the first configuration. The configuration of the ultrasonic transducers in then changed to the second configuration for imaging the internal body site. The subject devices and methods for their use find application in imaging a variety of different internal body sites.

### BRIEF DESCRIPTION OF THE FIGURES

FIGS. 1A to 1D provide representations of a first embodiment of the invention showing a multi-channel balloon. This balloon, when expanded, assumes a flat shape, thereby unfolding two transducer surfaces to form an effectively single, wide transducer.

FIGS. 2A to 2D provide representations of a second embodiment of the subject invention showing a balloon with a plurality of transducers, which connect together to form a single surface transducer when the balloon is inflated.

FIGS. 3A and 3B provide a representation of yet another embodiment of the subject invention showing a catheter with the ability to assume a pigtail shape (e.g., via shape memory, a pull wire or other means). The loops of the pigtail are in one plane. On the loop of the pigtail, a plurality of ultrasound transducers are installed, which, together, produce a single ultrasound transducer surface.

FIGS. 4A and 4B provide a representation of another embodiment of the subject invention showing a catheter with multiple stacked transducer arrays hinged at their proximal end. When expanded, the ultrasound array configuration produces a single compound transducer.

FIGS. 5A and 5B provide a representation of another embodiment of the subject invention in which a transducer array present on a flexible substrate, e.g., planar film, rests on an expandable side balloon positioned at the distal end of a catheter, where upon expansion of the side balloon, the array goes from a first to a second configuration, in which the second configuration is characterized by having a wider axial aperture than the first configuration.

FIG. 6A provides a representation of another embodiment of the subject invention showing a catheter with multiple hinged segments each having multiple transducer arrays. FIG. 6B provides a depiction of the device shown in FIG. 6A, where the device is in a folded configuration to produce a single compound transducer.

FIG. 7A provides a depiction of the another embodiment of the subject invention, showing an end view of a catheter with a configuration of multiple hinged elements each having multiple transducer arrays. FIG. 7B provides a side view of the catheter device depicted in FIG. 7A. FIG. 7C provides a depiction of FIG. 7A in a folded configuration to yield a single compound transducer.

FIG. 8A provides a representation of yet another embodiment of the subject invention in which an ultrasound array can be expanded and then rotated within a protective guard. FIG. 8B provides a view of the catheter depicted in FIG. 8A with the ultrasound array in an un-expanded condition. FIG. 8C provides an end view of the catheter depicted in FIG. 8A.

FIGS. 9A and 9B provide representations of yet another embodiment of the subject invention.

FIGS. 10A and 10B provide representations of yet another embodiment of the subject invention.

FIGS. 11A and 11B provide representations of yet another embodiment of the subject invention.

FIGS. 12A and 12B provide representations of yet another embodiment of the subject invention.

### DESCRIPTION OF THE SPECIFIC EMBODIMENTS

Devices and methods for obtaining a three-dimensional (3-D) image of an internal body site are provided. The subject devices are elongated structures (e.g., catheters) having a plurality of ultrasonic transducers located at their distal end. The configuration of the plurality of ultrasonic transducers may be reversibly changed from a first to a second configuration, where the radial aperture of the plurality of ultrasonic transducers is greater in the second configuration than in the first configuration. A feature of certain embodiments of the subject invention is that the plurality of ultrasonic tranducers are configured in the second configuration as a substantially continuous set of transducers. In using the subject imaging devices, the distal end of the devices is positioned at the internal body site of interest while the plurality of ultrasonic transducers is in the first configuration. The configuration of the ultrasonic transducers in then changed to the second configuration for imaging the internal body site. The subject devices and methods for their use find application in imaging a variety of different internal body sites.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, the present invention provides devices and methods, as well as systems and kits thereof, for obtaining a three-dimensional image of an internal body site. In further describing the subject invention, the subject devices are reviewed first in greater detail, followed by a more in-depth description of representative embodiments of the methods in which the subject devices are employed, as well as a review of various representative systems and kits that include the subject devices.

Devices

As summarized above, the subject invention provides ultrasound imaging devices that can be used in 3-D imaging of an internal body site. By “3-D imaging” is meant that the subject devices provide images that extend in three-dimensions, i.e., in the X, Y and Z planes. In other words, the subject imaging devices may be used to provide a volumetric image of an internal body site. A feature of the subject devices is that they can provide the 3-D image of the internal body site without having to construct the 3-D image from multiple 2-D images. As such, the subject devices can be employed to obtain 3-D images of an internal body site in real time (e.g., a four dimension image or 4-D image), where the provided 3-D images are not images reconstructed from multiple 2-D images, e.g., a series of 2-D images taken from different transducer locations in the internal body site being imaged.

To provide for the 3-D images during use, the imaging element of the imaging devices (e.g., compound transducer, as described in greater detail below) has a wide radial and axial aperture. The term “radial aperture” refers to the ultrasonic imaging window extending radially in any direction from the axis of the catheter body. The term “axial aperture” refers to the ulstrasonic imaging window extending longitudinally along the axis of the catheter body. As the imaging element, i.e., compound ultrasonic array transducer, has a wide radial and axial aperture, in certain embodiments the radial aperture typically ranges from about 1 to about 40 mm, such as from about 2 to about 30 mm, including from about 5 to about 20 mm, e.g., from about 1 to about 20 mm; while the axial aperture typically ranges from about 1 to about 40 mm, such as from about 2 to about 30 mm, including from about 5 to about 20 mm, e.g., from about 1 to about 20 mm.

In representative embodiments, the subject devices are elongate bodies (with a longitudinal and a radial axis) having proximal and distal ends. In many embodiments, the subject elongate devices are catheter devices. The catheter body is generally composed of a biologically compatible material that provides both structural integrity to the imaging catheter, as well as a smooth outer surface for ease in axial movement through a patient's body passage (e.g., the vascular system) with minimal friction. Such materials are typically made from natural or synthetic polymers, such as, e.g., silicone, rubber, natural rubber, polyethylene, polyvinylchloride, polyurethanes, polyesters, polytetrafluoroethylenes (PTFE) and the like. The catheter body may be formed as a composite having a reinforcement material incorporated within the polymeric body in order to enhance its strength, flexibility, and durability. Suitable enforcement layers include wire mesh layers, and the like. The flexible tubular elements of the catheter body may conveniently be produced by extrusion. If desired, the catheter diameter can be modified by heat expansion and shrinkage using conventional techniques.

The dimensions of the elongate body may vary considerably, e.g., depending on the particular target internal body site to be images, but in many embodiments the elongated tubular member is sufficiently long to provide for access of the distal end to the target body site upon introduction into the host vascular system via a remote entry site of the vascular system. Typically, for cardiovascular organs/sites, the length of elongate member ranges from about 90 to about 210 cm, such as from about 100 to about 190 cm and including from about 110 to about 150 cm. In yet other embodiments, e.g., for non-cardiovascular organs, catheter lengths may be less than about 90 cm, such as less than about 20 cm, but will in many embodiments be greater than about 5 cm, e.g., such as greater than about 10 cm. The outer diameter of the tubular member is such that it may be slidably moved in positioning the distal end of the device at the target site, and may range from about 1 to about 15 Fr, including from about 1 to about 12 Fr.

A feature of the subject imaging devices is that, located at the distal end of the devices, is a plurality of individual transducer elements that may be reversibly reconfigured or changed from a first configuration (format) to a second configuration (format). By plurality is meant at least 2, including at least about 5, such as at least about 10, where the number in the plurality can be as great as about 16, about 24 or more, and in many embodiments ranges from about 1 to about 500, such as from about 5 to about 300, including from about 10 to about 256.

The individual ultrasonic transducer elements may vary, as is known in the art, where representative ultrasonic transducer elements that may be employed include those described in US patents: U.S. Pat. Nos. 6,494,843; 6,482,162; 6,306,096; 6,171,247; 6,162,175; 6,129,672; 6,099,475; 6,039,693; 5,876,345 and 5,713,363; as well as published U.S. patent application Ser. No. 2002/0026118 A1; the disclosures of which are herein incorporated by reference. In representative embodiments, the transducer elements are fabricated from piezoelectric or silicon materials, as is known in the art.

As indicated above, a feature the subject devices is that the distally located or positioned plurality of transducer elements is one that can be reversibly configured from a first format or configuration to a second format or configuration. As such, the spatial arrangement of the plurality of transducer elements can be reversibly changed from a first pattern to a second pattern.

The second configuration is distinguished from the first configuration by having a wider radial aperture than the radial aperture of the first configuration. In representative embodiments, the radial aperture of the second configuration is at least about 2 to about 20 times, such as at least about 2 to about 20 times, including at least about 2 to about 5 times wider than the radial aperture of the first configuration. By “reversibly” is meant that the plurality of the ultrasonic transducer elements can be changed from the first to second configuration and then back to the first configuration as desired, e.g., as commanded by the operator of the imaging device. As such, the plurality of transducer elements can be readily reconfigured between the first and second configurations as desired.

The plurality of transducer elements is further characterized in that the first configuration provides for a distal end outer diameter of the device that is shorter than the distal end outer diameter of the device when the transducer elements are present in the second configuration. The magnitude of difference in length of the outer diameter between the first and second configurations in many embodiments is at least about 2-fold, such as at least about 3, 4, 5 fold or more. The outer diameter in the first configuration in certain embodiments ranges from 1 to about 15 Fr, including from about 1 to about 12 Fr; and from about 5 to about 70 Fr, such as from about 10 to about 50 Fr, in the second configuration. The shorter outer diameter in the first configuration provides for a “low catheter profile” during introduction of the distal end of the imaging device to the target body site to be imaged.

A feature of certain embodiments of the subject invention is that the plurality of ultrasonic tranducers are configured in the second configuration as a substantially continuous set of transducers. As such, at least in the second configuration, the set or multitude of transducers assumes a configuration that is not a “sparse” array of transducers, as is known in the art. Instead, the multitude or set of transducers is configured in a manner that provides an effective single transducer. Accordingly, the image acquired from the set or plurality of transducers when present in the second configuration need not be interpolated, as is done when using “sparse” array configurations. In certain embodiments, any given transducer of the plurality is touching at least one other transducer in the plurality such that a continuous linear configuration of transducers is provided in the second configuration. If any space is present between transducers in this continuous linear configuration, such space does not exceed about 5 transducers widths in length, such as about 3 transducer widths in length, including about 1 transducer width in length, where a transducer width is the average transducer width of all of the transducers in the array. In this manner, the transducers provide an “effective” single transducer in the larger radial aperture of the second configuration.

A feature of certain embodiments is that the distances between at least a portion or subset of the individual transducers, e.g., at least about 10%, at least about 20%, at least about 30%, at least about 40%, at least about 50%, at least about 60%, or more, including at least about 75%, 80% or 90% or even all of the transducers, does not change during transition between the first and second configurations. In these embodiments, the transition from the first to the second configuration simply re-orients the transducers relative to each other, but does not spread them apart from each other, to face a desired sonication surface.

The imaging devices may have a variety of different configurations or structures, where representative configurations are further described below in view of the figures.

As is known in the art and described in the above patents and publications listed under the “Relevant Literature” heading, the subject imaging devices may further include a number of additional elements as desired, e.g., circuitry to convey electrical signals between the transducer elements and a processor, e.g., where the processor may be located external to the patient or subject being imaged or at or near the proximal end of the device, e.g., close to the transducers; one or more additional lumens with access ports for introducing additional tools (e.g., tissue ablators, sensors, therapeutic agent delivery members, etc.) to the target site; deflection or steering features and the like.

In certain embodiments, the devices may include a mechanical placement element that positions the transducer array in a desired three-dimensional space upon deployment and during use. For example, the catheter device may further include a balloon or cage at the distal end that deploys upon placement at least proximal to the target site and in which the array is positioned upon deployment, where the array then images the target site from which the balloon or cage (or analogous structure).

In certain embodiments, also provided are mechanisms that provide for reliable transition between the first and second configurations of the device, e.g., to ensure proper unfolding and retrieval of the device from the target site being imaged. Such mechanisms include, but are not limited to: one or more spring elements to hold the transducers of the array in a predetermined alignment; hinges that prevent motion beyond a desired position (e.g., inter-digitating hinges); circuit-closure and analogous detection elements to sense angle and planarity; optical encoders to measure angles, etc.

The subject imaging devices having now been generally described, the subject devices will now be further described in view of several representative embodiments as depicted in the Figures.

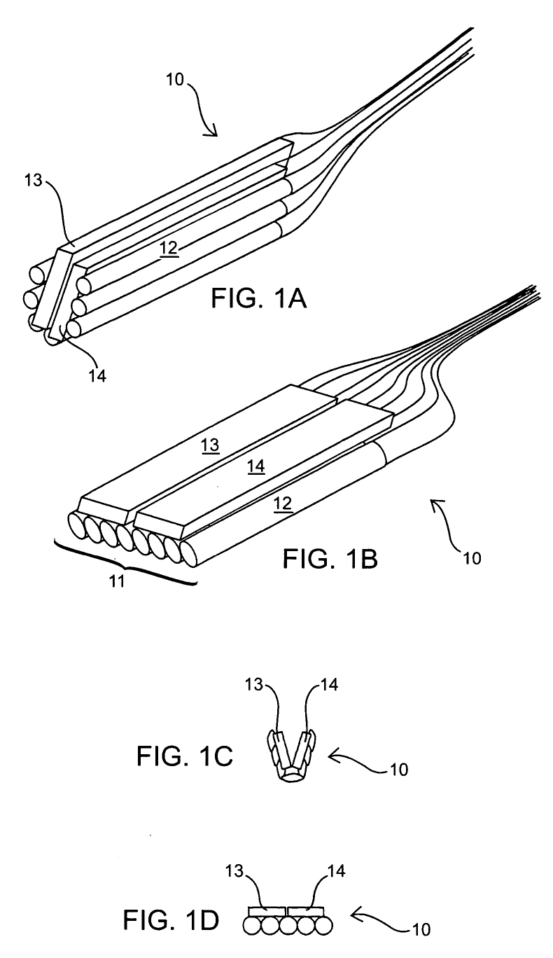

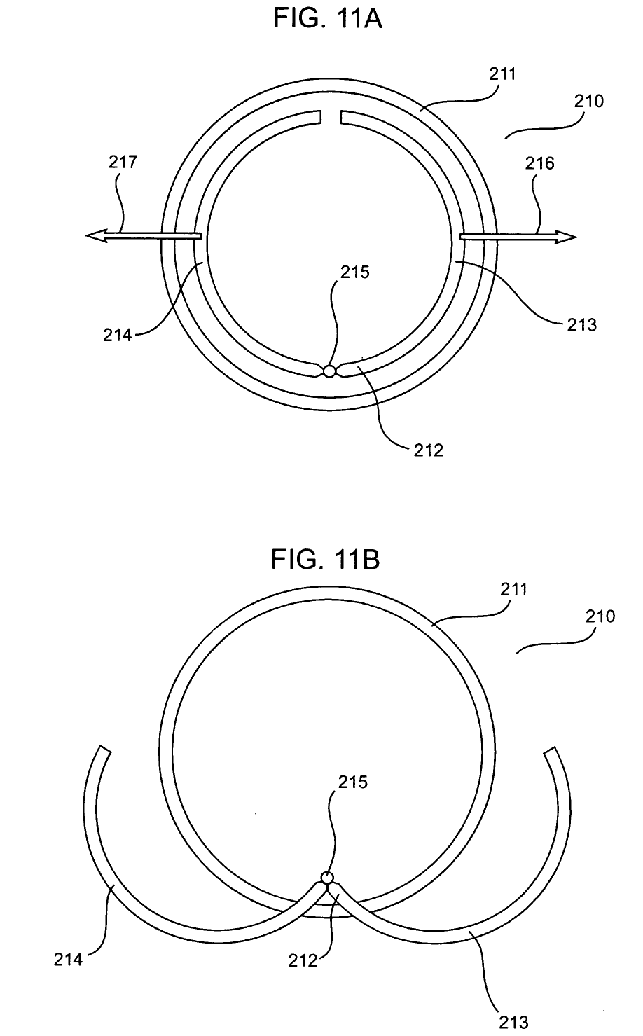

FIG. 1 provides a depiction of a representative embodiment of the subject imaging devices. In this embodiment, a catheter having a distal tip that can be introduced into a bodily cavity is employed. At the distal tip of the catheter, an imaging element 10 having a hydraulically inflatable balloon 11 with multiple parallel inflatable channels 12 is installed. In a first configuration, the balloon is folded in a manner to create a pocket, as depicted in FIGS. 1A and 1C. When inflated in the second configuration, the balloon unfolds to produce a straight flat surface of the ultrasound transducers 13 and 14. A plurality of elongated ultrasound arrays can be attached to the balloon in parallel fashion. In folded position, e.g., as shown in FIGS. 1A and 1C, the balloon pocket would house the ultrasound arrays in parallel fashion. In inflated position, e.g., as shown in FIGS. 1B and 1D, the flat balloon would unfold the array configuration to form a compound ultrasound transducer with desired aperture width, especially in radial direction. As shown in FIGS. 1B and 1D, transducers 13 and 14 touch each other in the deployed configuration to produce a continuous transducer structure along the radial aperture of the second configuration. In both of these representative embodiments, the plurality of transducer elements at the distal end of the device is reversibly reconfigurable from a first to a second configuration, where the first configuration has a low-profile for ease of introduction to the target body site and the second configuration has a wide radial aperture (that is wider than the radial aperture of the first configuration) for imaging the body site during use. In the second configuration, the plurality of transducer elements form a 2-D array, while in the first configuration they do not. In addition, both representative embodiments are characterized by having a distal end inflatable balloon which provides for the reversibly reconfigurable nature of the plurality of transducer elements. It is noted that the imaging structure depicted in FIG. 11 is shown as having two elongated transducers, 13 and 14. However, devices of this embodiment may have more than two elongated transducers, e.g., three or more, such as four or more or even five or more, where in the first configuration the elongate transducers may assume a stacked configuration so as to provide the desired low profile during positioning.

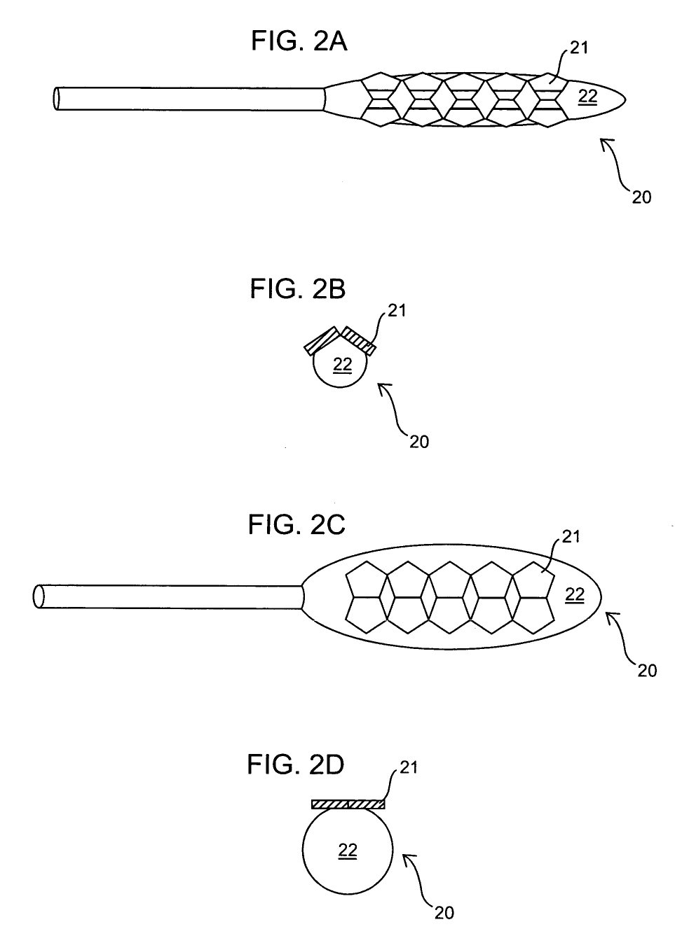

FIGS. 2A to 2D show another representative embodiment of a catheter imaging device according to the present invention. In this embodiment, positioned at the distal tip of a catheter is an imaging element 20, which imaging element 20 includes a plurality of ultrasound elements (e.g., piezoelectric crystals, silicon) 21 installed in a configuration allowing the individual transducers to change orientation of the sonication surface of the element, e.g., to form a single compound transducer during function. The ultrasound elements are attached to a hydraulically inflatable balloon 22 located beneath the sonication elements 21. In the inflated position (as shown in FIGS. 2C and 2D), the balloon aligns the ultrasound elements 21 so that a single (compound) transducer array is formed, as shown in FIGS. 2C and 2D. The resulting compound transducer provides a desired aperture width, especially in radial direction, in a manner analogous to the embodiment depicted in FIGS. 1A to 1D.

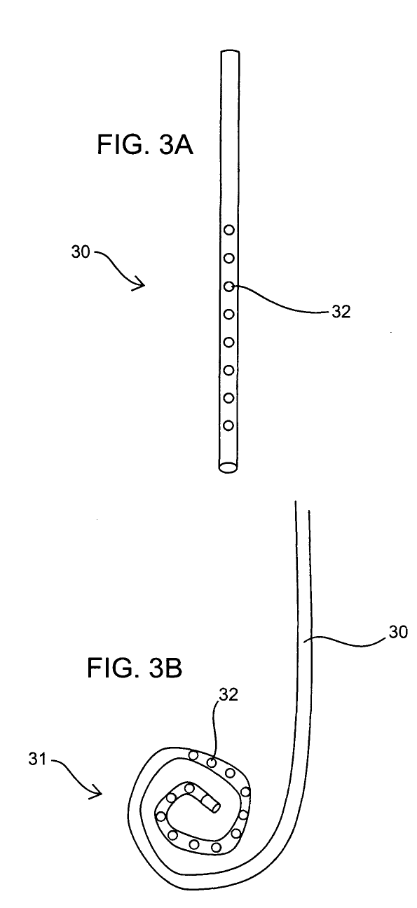

FIGS. 3A and 3B depicts yet another representative embodiment of the present invention. FIG. 3A shows the first configuration and FIG. 3B shows the second configuration of this embodiment. A catheter 30 with pig-tail shape, which can be straightened during introduction into target body site, e.g., body cavity, is shown. The loops of the pigtail 31, as shown in FIG. 3B, are designed to position the plurality of transducer elements into one single 2-D plane upon placement of the distal end at the target site. A plurality of ultrasound transducers 32 is on the exterior surface of the catheter, with the sonication surface facing in the same plane. As such, upon introduction, the individual ultrasound elements align into a compound ultrasound transducer surface with the desired aperture width. In the embodiment, the transducers 32 are shown spaced apart from each other on the surface of the catheter. However, in certain embodiments, the transducers are positioned adjacent to each other such, a continuous line of transducers is provided.

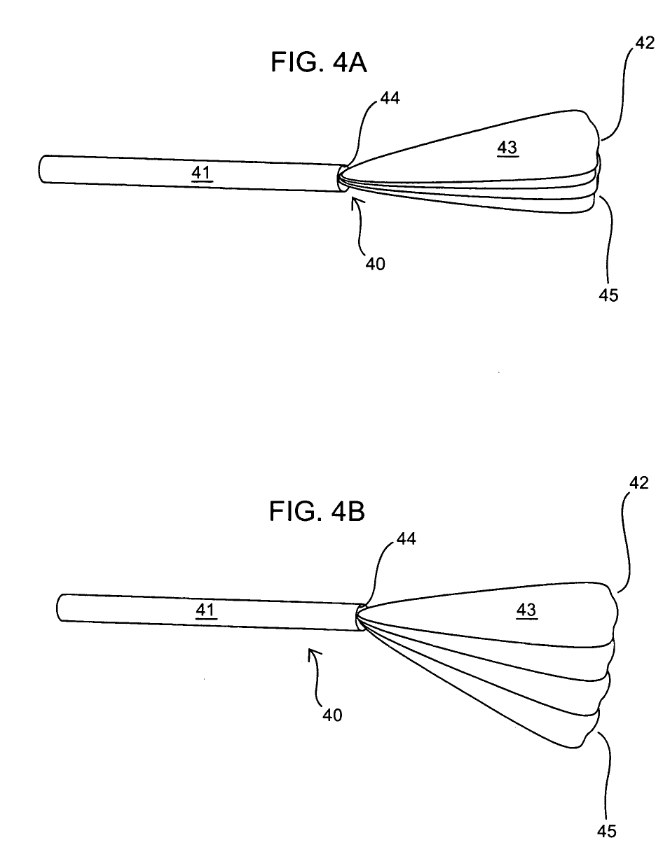

FIGS. 4A and 4B show yet another embodiment 40 of the subject invention, where a catheter structure 41 has and imaging element 42 made up of multiple stacked transducer arrays hinged at their proximal end 44. During operation, the stacked ultrasound transducer arrays are diverged at their distal end 45, thereby forming a single compound transducer with desired aperture width, especially in radial direction.

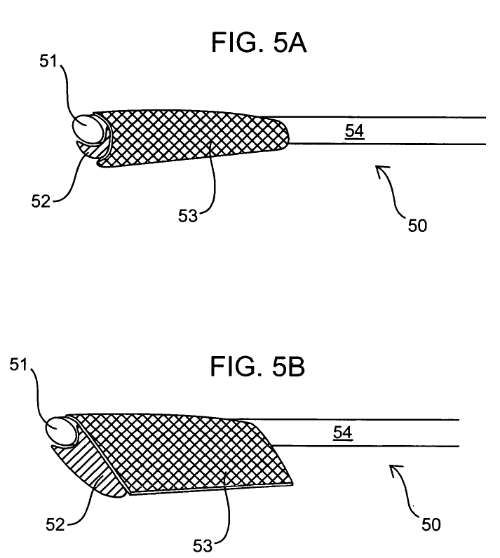

FIGS. 5A and 5B provide a representation of yet another balloon embodiment of the subject invention in which a transducer array 53 present on a flexible substrate, e.g., planar film, rests on an expandable side balloon 52 positioned at the distal end 51 of a catheter 54. Upon expansion of the side balloon, the array goes from a first configuration (as shown in FIG. 5A) to a second configuration (as shown in FIG. 5B), in which the second configuration is characterized by having a wider radial aperture than the first configuration.

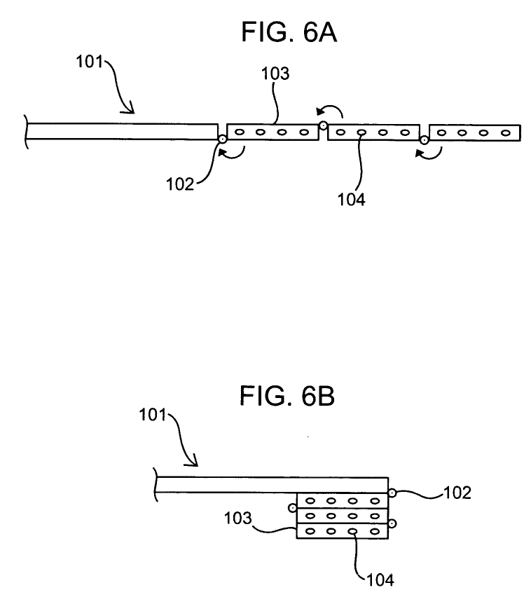

FIG. 6A depicts yet another representative embodiment of the subject invention in which individual ultrasound elements 104 are disposed along multiple catheter segments 103 which can be folded to form a compound array as shown in FIG. 6B. The multiple catheter segments 103 are connected by hinge elements 102 which alternate sides to allow folding as depicted by arrows in FIG. 6A. The hinge elements 102 may be constructed using a variety of methods. For example these may be similar to a door hinge, or may be thinned areas separating the multiple catheter segments 103, or other suitable constructions. Means for actively causing folding of the multiple catheter segments 103 (pull wires, for example, not shown) may also be incorporated into the device. Alternatively, the multiple catheter segments 103 may be predisposed to assume a folded configuration and may be selectively constrained in a straight configuration, as by a sheath (not shown) or a stiffening member (also not shown) for the purpose of introduction. Further, the hinge elements 102 may be adapted to enable transmission of signals to and from ultrasound elements 104 disposed on the device.

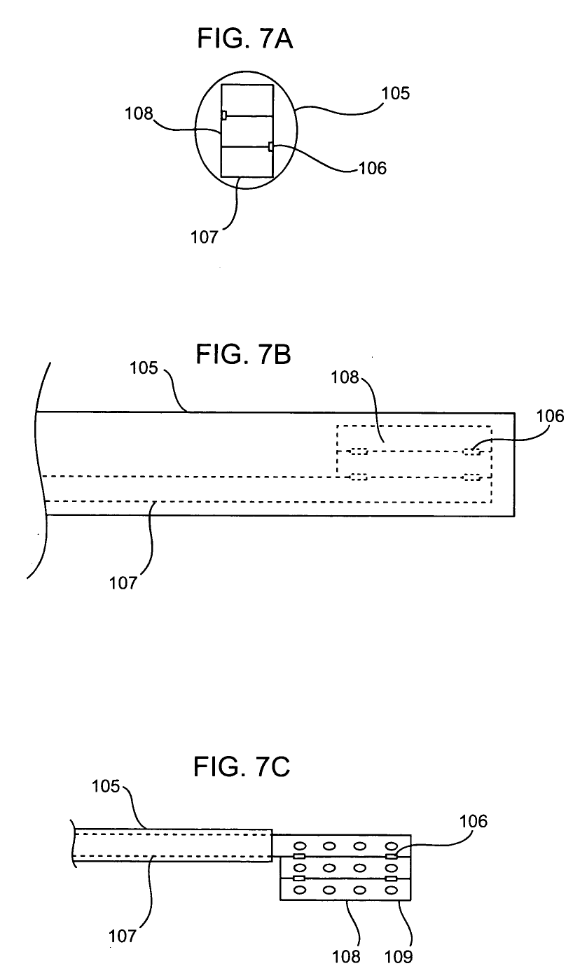

FIGS. 7A, 7B and 7C depict a related configuration in which multiple catheter segments 108 fold in a horizontal direction rather than the longitudinal direction depicted in FIGS. 6A and 6B. This folding array may be delivered in a sheath 108 and advanced out of the sheath prior to unfolding of the multiple catheter segments 108.

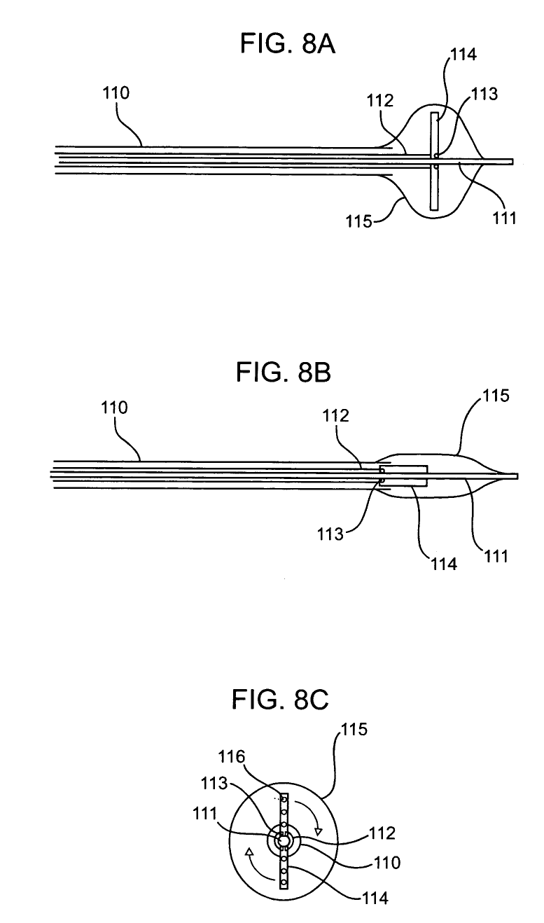

Another representative embodiment for constructing a compound ultrasound array is depicted in FIGS. 8A to 8C. Here, multiple ultrasound elements 116 are disposed on one or more unfolding segments 114. These unfolding segments lay fiat as depicted in FIG. 8B until advanced beyond the distal end of the catheter body 110. Once advanced beyond this point, the segments 114 may actively or passively deploy into an expanded position as depicted in FIGS. 8A and 8C. Unfolding into and expanded position may be facilitated by hinge elements 113. Further, unfolding segments 114 may be rotated by rotating an inner member 112 to which they are connected. This rotation may be used to create an effective disc shaped transducer array by temporally resolving images obtained at multiple positions through the rotation path. Additionally, a protective means 115 may be disposed around the rotating segments 114 to protect the structures and/or tissue which are being imaged and to enable rotation of the segments 114 in a controlled manner. The protective means 115 may be expandable, such as an inflatable balloon or an expandable wire basket, or an expandable mesh tube, or may be constructed using other suitable means.

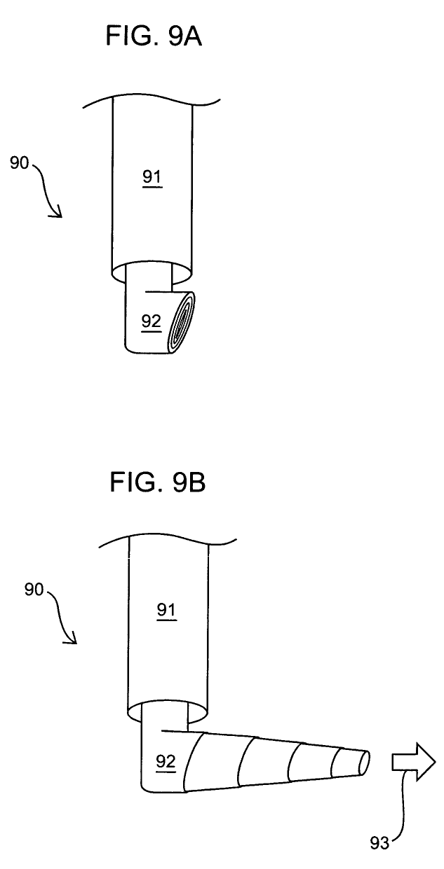

Yet another embodiment of the subject invention is depicted in FIGS. 9A and 9B. FIG. 9A shows the device in the first configuration while FIG. 9B shows the device in the second configuration. Device 90 includes elongated catheter body 91 with imaging element 92 positioned at the distal end. Imaging element is structured in a spirally wound configuration that can be extended in a sidewise direction (as shown by arrow 93) upon deployment. While the embodiment shown in FIGS. 9A and 9B expands in only one direction, in certain embodiments the device a bi or multilateral device that expands in two or more radial directions from the longitudinal axis of the catheter. Upon expansion spirally wound imaging element, the array present thereon goes from a first configuration (as shown in FIG. 9A) to a second configuration (as shown in FIG. 9B), in which the second configuration is characterized by having a wider radial aperture than the first configuration.

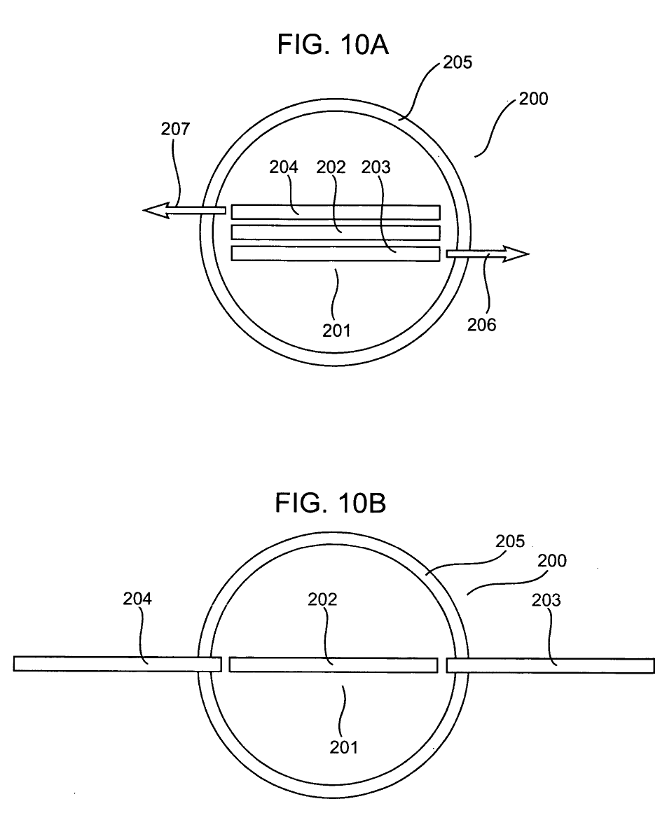

Yet another embodiment of the subject invention is depicted in FIGS. 10A and 10B. FIG. 10A shows the device in the first configuration while FIG. 10B shows the device in the second configuration. Both FIGS. 10A and 10B provide end-views of the distal end of catheter device 200. Device 200 includes elongated catheter body 205 with imaging element 201 positioned at the distal end. Imaging element is a stacked structure of planar transducers 202, 203 and 204. Upon deployment, the imaging element is first moved longitudinally out of the distal end of catheter body 200. Transducers 203 and 204 are then extended in a sidewise direction (as shown by arrows 206 and 207 respectively) upon deployment, such that they assume a planar configuration. Upon deployment of the imaging element, the array present thereon goes from a first configuration (as shown in FIG. 10A) to a second configuration (as shown in FIG. 10B), in which the second configuration is characterized by having a wider radial aperture than the first configuration.

While the above specific embodiments have been described in terms of providing a 2D array of transducers, in which all of the transducers lie in a single plane upon deployment of the imaging device from a first to a second configuration, also provided are embodiments that provide for a 3-dimensional array of transducers upon deployment of the imaging device. An example of such an embodiment is shown in FIGS. 11A and 11B. FIG. 11A shows the device in the first configuration while FIG. 11B shows the device in the second configuration. Both FIGS. 11A and 11B provide end-views of the distal end of catheter device 210. Device 210 includes elongated catheter body 211 with imaging element 212 positioned at the distal end. Imaging element 212 is made up of two curved planar structures 213 and 214 joined by hinge element 215. Upon deployment, the imaging element is first moved longitudinally out of the distal end of catheter body 210. Transducers 213 and 214 are then extended in a sidewise direction (as shown by arrows 216 and 217 respectively) upon deployment, such that they assume a deployed configuration. Upon deployment of the imaging element, the array present thereon goes from a first configuration (as shown in FIG. 11A) to a second configuration (as shown in FIG. 11B), in which the second configuration is characterized by having a wider radial aperture than the first configuration. A feature of the second configuration is that the transducer array is a 3-dimensional transducer array.

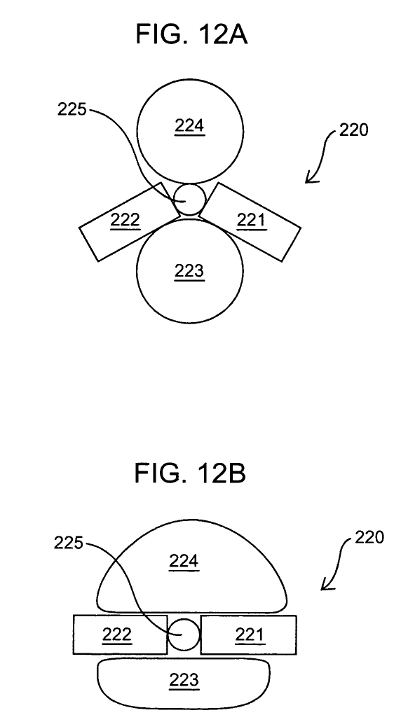

Yet another embodiment of the subject invention is depicted in FIGS. 12A and 12B. The embodiment shown in FIGS. 12A and 12B is a various of the devices shown in FIGS. 1 and 2. FIG. 12A shows the device in the first configuration while FIG. 12B shows the device in the second configuration. Both FIGS. 12A and 12B provide end-views of imaging element 220 which can be positioned at the distal end of a catheter body. Imaging element 220 includes two planar transducers 221 and 222 joined by hinged element 225 and sandwiched between first and second balloons 223 and 224, respectively. Upon deployment via inflation of balloons 223 and 224, transducers 221 and 222 assume a planar configuration. Upon deployment of the imaging element, the array present thereon goes from a first configuration (as shown in FIG. 12A) to a second configuration (as shown in FIG. 12B), in which the second configuration is characterized by having a wider radial aperture than the first configuration.

In sum and as described both generally and in view of several specific representative embodiments, the present invention provides an imaging device, e.g., catheter, that is characterized by having plurality of transducing elements which can be reversibly reconfigured from a first to second configuration, where the second configuration has a wider radial aperture than the first configuration and, at least in many embodiments, provides a compound transducer array.

Methods

Also provided are methods of using the subject imaging devices. The subject methods are typically imaging methods, where an internal body site of a subject is to be imaged. In representative embodiments, the subject devices are employed to image an internal body site of a mammal, where this term is used broadly to describe organisms which are within the class mammalian, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the animals or hosts, i.e., subjects (also referred to herein as patients) will be humans.

In practicing the subject methods, the distal end of an imaging device according to the present invention is positioned at least proximal to, e.g. near or at the target internal body site to be imaged, e.g., using standard protocols. The internal body site may be any of a variety of different body sites, including, but not limited to: intracardiac, intravascular, and extravascular structures, such as cardiovascular body sites, such as a chamber of the heart, an arterial site, abdominal and urogenital cavities, and the like; as well as other internal body sites.

Upon positioning of the distal end of the imaging device at least proximal to the target internal body site, the configuration of the plurality of ultrasound transducer elements is then reconfigured or changed from the first to second configuration, where the particular protocol employed in this reconfiguration step necessarily depends on the nature of the specific device being employed.

Once present in the second configuration, the resultant compound transducer array is then employed to image the site, using protocols known in the art, including protocols in which the transducer element is mechanically moved during imaging, protocols in which the transducers are phase activated, etc. Because of the structure of the compound array in the second configuration, a real time 3-D image of the internal body site may be obtained, as desired.

In certain embodiments, the methods may include an image data processing step, in which the orientation of the transducer elements in 3-dimensional space is determined and, if needed, the obtained signal is corrected as desired to account for any variability arising from the particular detected orientation of the elements. For example, sensor elements on the device, as well as the elements themselves, may first be employed to determine whether the array in the second configuration assumes a planar or non-planar structure. If a non-planar structure is detected, the collected image data may then be processed to correct for this non-planar structure, e.g., using suitable algorithms that are readily determined by those of skill in the art. As such, the methods may include use of devices that monitor (1) transducer expansion state, and (2) variability in element performance during operation, as well as means for correcting data, e.g., images and measurements, in case of “suboptimal” expansion of transducer and/or array element irregular performance.

Upon completion of imaging of the internal body site, the configuration of plurality of transducers is returned to the first configuration, and the device removed from patient or subject.

The above methods provide an image, and in many embodiments and 3-D image, of a target internal body site. A feature of the subject methods is that they can provide a real time 3-D image of the internal body site, which need not be reconstructed from a plurality of 2-D images taken at different times.

Utility

The subject invention finds use in any application where accurate imaging of an internal body site, and particularly where accurate 3-D imaging of an internal body site, is desired. Such applications include, but are not limited to: those described in US patents: U.S. Pat. Nos. 6,494,843; 6,482,162; 6,306,096; 6,171,247; 6,162,175; 6,129,672; 6,099,475; 6,039,693; 5,876,345 and 5,713,363; as well as published U.S. patent application Ser. No. 2002/0026118 A1; the disclosures of which are herein incorporated by reference. Two representative applications in which the subject imaging methods and devices find use are diagnostic and interventional applications.

For example, the subject methods and devices can effectively perform diagnostic intracardiac and transvascular imaging. Such applications may typically be performed just prior to an interventional application. Some specific examples of diagnostic imaging include, but are not limited to: 1) accurate visualization and measurement of an intracardiac defect; 2) characterization of valve orifices; 3) localization of a tumor; and the like. Extravascular diagnoses may include, but are not limited to: 1) visualization of pancreatic mass/pathology; 2) visualization of retroperitoneal pathology; 3) intracranial imaging; 4) recognition of perivascular pathology; 5) imaging of other internal body spaces such as urinary bladder, bile system, fluid filled orifice or cavity (e.g. filled saline), etc.

The subject devices and methods may also be employed during interventional applications, where imaging using the subject methods and devices is employed together with another technology, such as: 1) an occlusion device for closure of a wail defect; 2) an ablation catheter for treatment of arrhythmia; 3) a blade septostomy catheter or laser-based catheter system to produce a desired defect; 4) devices employed in cardiovascular anatomic repair procedures (such as valve repair and implantation, cardiac appendage reconstruction, etc), 5) Others (such as prostrate surgery, placement of stents, gallstone removal etc.); etc. By direct imaging of an application, such as ablation, a procedure can be performed more safely and repeatedly, and the result can be better assessed.

Systems

Also provided are systems for use in practicing the subject methods, where the systems at least include an imaging device, as described above. The subject systems also typically at least include an external ultrasound processing element or means, which element or means is capable of electrically communicating with the transducer elements to produce a 3-D image according to the subject invention. The subject systems may also include, where desired, transducer array monitoring elements, e.g., to determine the configuration of the elements in three-dimensional space, and imaged data processing elements, e.g., software, as described above. In addition, in many embodiments the systems also include one or more additional elements, e.g., elements finding use in interventional applications, balloon inflation means, etc.

Kits

Also provided are kits for use in practicing the subject methods, where the kits typically include one or more of the above devices, and/or components of the subject systems, as described above. As such, a representative kit may include a device, such as a catheter device, as described above. The kit may further include other components, e.g., guidewires, interventional devices, etc., which may find use in practicing the subject methods.

In addition to the above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

It is evident from the above description that the subject invention provides a significantly improved method of obtaining a 3-D image of an internal body site. Because of the nature of the subject devices, radially wide 3-D images can be obtained in real time from a device that has a low profile during introduction to the body site of interest. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. An ultrasound imaging device comprising:
(a) an elongate body having a longitudinal axis, a distal end and a proximal end; and
(b) a plurality of individual ultrasonic transducers positioned at said distal end that can be changed from a first configuration to a second configuration, wherein said second configuration provides for a substantially continuous array of ultrasonic transducers that has a radial aperture that is wider than said first configuration.

2. The catheter according to claim 1, wherein said plurality of individual ultrasonic transducers is positioned in a two-dimensional array in said second configuration.

3. The catheter according to claim 2, where said plurality of individual ultrasonic transducers is not positioned in a two-dimensional array in said first configuration.

4. The catheter according to claim 3, wherein said plurality of individual transducers is positioned in a linear arrangement in said first configuration.

5. The catheter according to claim 1, wherein said distal end comprises an inflatable balloon having said plurality of ultrasonic transducers present thereon.

6. The catheter according to claim 5, wherein said inflatable balloon is a multiple chambered balloon that assumes a flat balloon configuration upon deployment.

7. The catheter according to claim 5, wherein said inflatable balloon is a rounded balloon.

8. The catheter according to claim 1, wherein said distal end is linear in said first configuration and is curled upon itself in said second configuration to produce said a two-dimensional array of ultrasonic transducers.

9. The catheter according to claim 1, wherein said distal end comprises stacked arrays.

10. The catheter according to claim 1, wherein said distal end comprises at least one hinge.

11. The catheter according to claim 1, wherein said distal end has a diameter that is larger in said second configuration than in said first configuration.

12. The catheter according to claim 1, wherein said plurality of transducers is present in a mechanical placement element.

13. The catheter according to claim 12, wherein said mechanical placement element is a balloon or cage.

14. The catheter according to claim 1, wherein said plurality of individual ultrasonic transducers in said second configuration is capable of providing a 3-D image of an object without external reconstruction of 2-D images.

15. A method of imaging an internal body site, said method comprising:
(a) positioning a distal end of a catheter according to claim 1 at least proximal to said internal body site, wherein said plurality of ultrasonic transducers is present in said first configuration;
(b) changing said plurality of ultrasonic transducers to said second configuration; and
(c) imaging said internal body site using said plurality of ultrasonic transducers in said second configuration.

16. The method according to claim 13, wherein said method provides a 3-D image of said internal body site without longitudinal movement of said distal end of said catheter.

17. The method according to claim 13, wherein said method further comprises mechanically moving said plurality of transducers during said imaging step (c).

18. A system for imaging an internal body site, said system comprising:
(a) a catheter according to claim 1; and
(b) an ultrasound data processing element for processing data obtained from said plurality of transducer elements.

19. The system according to claim 18, wherein said processing element provides a 3-D image of said internal body-site using data obtained from said catheter.

20. A kit for imaging an internal body site, said kit comprising:
(a) a catheter according to claim 1; and
(b) instructions for using said catheter in an internal body site imaging application.

21. The system according to claim 20, wherein said system further comprises an image data processing element for correcting image data.
